(19) United States
(12) Patent Application Publication
(10) Pub. No.: US 2006/0254598 A1
(43) Pub. Date: Nov. 16, 2006

(54) Caesarean belt

(76) Inventor: Oliver Saul, Hennef (DE)

Correspondence Address: Theraline LLC, 29 Locksvillage Road, Wendell, MA, 01379, US

(21) Appl. No.: 11/415,494

(22) Filed: May 2, 2006

Related U.S. Application Data
(60) Provisional application No. 60/680,964, filed on May 12, 2005.

(51) Int. Cl. A61F 5/00; A61F 5/37

(52) U.S. Cl. 128/876; 602/19

(57) ABSTRACT

Belt to protect Cesarean incisions against kicking from infants, e.g. when breastfeeding or from accidental banging. By inserting warming and cooling cushions, therapeutic effects can also be achieved. The belt contains a pocket into which inserts with different functions can be placed depending upon the application. A plastic shell can be used to protect from kicking and other kinds of impact, and a polyester cushion if further absorption is required. In order to soothe uterus cramps and promote healing, a cherry stone cushion can be warmed and used and a gel cushion is recommended for cooling possibly, swollen, inflamed or itching scars.

### BACKGROUND

In Caesarean section seven layers of female body tissue are severed. The incision is usually made above the pubic hairline and is (vertically) about 4 inches long. This has the advantage that the person in question can wear a bikini or similar apparel again after Caesarean section and that the scar remains inconspicuous after the wound has healed. However, this area of the abdomen is also very sensitive.

If the child is breastfed with the head lying level with the breasts and/or lying stomach to stomach with the mother, then—depending upon the size of the baby—its feet are exactly level with the Caesarean section scar. As the baby's movements can be considerable depending on its predisposition, the problem arises that it kicks directly against the mother's scar during breastfeeding. Later when the child is larger, it could also be the legs or knees with which it “kicks into” the mother's Caesarean scar.

Many women report that they feel insecure for some time after the C-section and concerned about inadvertently bumping into something. Usually a hand is held in front of the wound either consciously or unconsciously as protection.

As mentioned above, during Caesarean section many layers of tissue are cut through. After approximately six weeks the wound has healed to a large extent, but it takes longer before the scar area has healed completely and is no longer painful. Especially in the first few weeks after the C-section, however, any kicking by the child in the pubic region or the freshly operated abdomen is very unpleasant and often painful, and/or strong kicks can cause long-lasting pain in sensitive individuals. Sometimes the scar becomes swollen again after a kick from the baby's feet. Scar healing can thus be delayed in extreme cases.

Furthermore, after Caesarean births women find normal uterine contractions during breastfeeding to be particularly painful.

In some cases, complications such as infections arise during scar healing. Unpleasant itching often occurs, for example. In many cases the elastic of the underwear or trousers belt rubs against the fresh scar.

### BRIEF DESCRIPTION OF THE INVENTION

The Caesarean section belt is a multifunctional aid, which helps to avoid or alleviate a multitude of the cited complaints arising in conjunction with a Caesarean section or other operations in the uterine area.

The pocket integrated in the belt enable various elements to be inserted, which have shock absorbing, cooling and/or warm effects.

The combination of an ergonomically adapted plastic shell and an additional absorbent cushion effectively protects the scar and the surrounding tissue against knocks, including those of a baby for instance, e.g. when carrying or breastfeeding, against riotous young siblings and accidental knocks.

Whether individually or in combination, the plastic shell and cushion also protect the healing scar from e.g. rubbing against the elastic band of underwear or the trouser waistband or belt. Furthermore, they also act as a protection against the seatbelt when travelling by car.

With the aid of the belt pocket, both applications with gel filling and cherry pit filling can be placed exactly over the scar and used cooled or warmed according to individual need.

Cooling ensues mainly in the case of swelling, infections or irritation. The warming effect relaxes abdominal cramps, which occur during uterine contractions or also relaxes in the case of menstruation or back pains.

### BRIEF DESCRIPTION OF THE DRAWINGS

FIG. 1 is a perspective view of one embodiment with button hole fastening system of a Cesarean Belt according to the invention.

FIG. 2 is a perspective view of a further embodiment with clip fastening system of a Cesarean Belt according to the invention.

FIG. 3 is a perspective view of another embodiment with Velcro fastening system of a Cesarean Belt according to the invention.

FIG. 4 is a front view of the cherry stone insert

FIG. 5 is a front view of the the cold-warm gelpack

FIG. 6 is a front view of the plastic shell

FIG. 7 isa front view of the polyester fiber pillow

### DETAILED DESCRIPTION OF THE INVENTION

In order to avoid the previously described problems and risks, a protective belt is recommended after a Caesarean operation as it is defined in the protective claims. In order to avoid the described troubles and risks and to achieve the desired protection and medicinal benefits, a protective belt as defined in the protective claims is recommended after a Caesarean section or other operation in the uterine area.

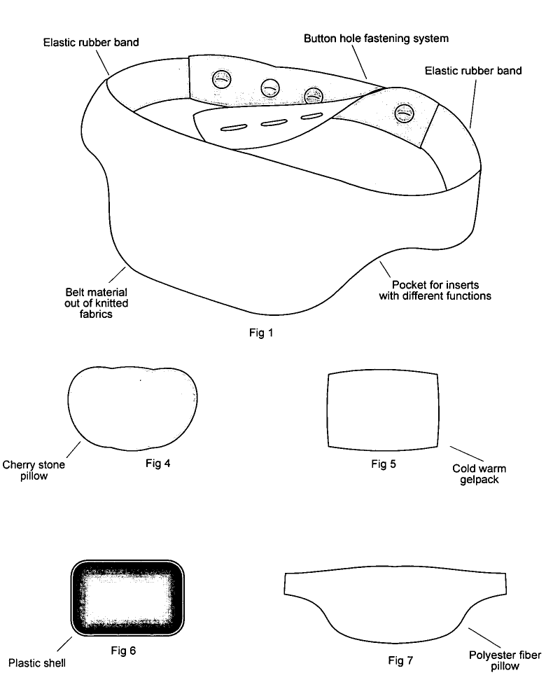

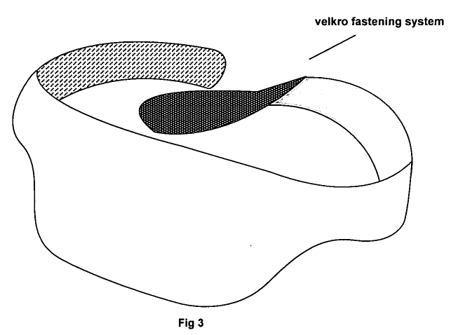

The Caesarean section belt FIG. 1 excels in that it is available in the form of a belt, which is able to hold several different impact absorbing elements individually or in combination (see FIG. 4 cherry stone cushion, FIG. 5 gel cushion, FIG. 7 polyester cushion, FIG. 6 plastic shell), firmly over the Caesarean scar.

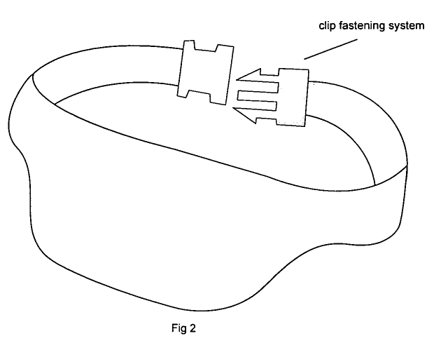

The belt can be equipped with 3 different fastening systems: Velcro (FIG. 2), clip (FIG. 3) or buttonhole fastening (FIG. 1), which each ensure an optimal fit and size.

When using the Velcro fastening system, FIG. 2, the overall length ranges from at least 31 inches to a maximum of 47 inches. The design ensues in various sizes: size S 31-39 inches, size M 35-43 inches and size L 39-47 inches. The buttonhole and clip fastening systems (FIGS. 3 & 4) have the advantage that only one size is necessary. The belt length in this instance is 41-47 inches, with the preferred length being 43 inches.

The impact absorbing elements are held in a pocket, which not only covers the Caesarean scar, but also the sensitive area above and below the scar. The pocket in the belt can be made of stretchable material like the belt itself.

In addition, particularly flexible elastic bands (see FIG. 1) are sewn into the belt part which provide a pleasant fit, e.g. when bending or in different body postures.

The expandable elastic bands range from at least 2 inches to 7 inches in length. The preferred design is 6 inches long.

The design of the belt is such that it can be worn comfortably e.g. just with the polyester cushion (FIG. 7) underneath the underwear, in order to avoid rubbing, e.g. of the elastic.

The belt pocket height ranges from at least 4 inches to 7 inches, with the preferred design being 6 inches in height, meaning that both the Caesarean section scar and the sensitive area around it are covered at the same time.

The area about 1 inch above and 1 inch below the scar is usually very sensitive to contact and pressure for months after birth and can also feel numb for up to two years after the operation.

In order to achieve effective protection against knocks and clothing, the use of a hard PVC plastic shell is preferable to that of a foam rubber cushion.

The above-cited plastic shell (FIG. 6) consists optionally of formed PVC, polypropylene or polyethylene. The preferred design is in PVC. The plastic shell dimensions are 6-8 inches in breadth and 3-5 inches in height. The preferred design is 7×4 inches.

The plastic shell is formed in such a way that it leaves a gap of approximately 1 inch between scar tissue and surrounding area in the middle area. The material is created in such a way that it ensures form stability, e.g. even in the case of powerful knocks or tightly fitting trouser belts.

Another particular advantage of the plastic plastic shell is that, despite its highly protective effect, it is only spread very thinly, meaning that the user can wear it very inconspicuously.

In order to buffer the relatively hard breadth, a polyester cushion may be inserted in the pocket between skin and breadth.

The polyester cushion may also be used to insulate the warmth or cold dispensing elements.

Furthermore, it may also be used alone as a protection against rubbing underwear.

The polyester cushion (FIG. 7) consists of polyester wadding clothed in cotton. The polyester wadding is 1-2 inches in thickness. The preferred design is 1 inch thick.

The height and length dimensions of the polyester cushion are reconciled so precisely with the belt pocket that it receives the optimal hold. The preferred dimensions are 14/7 inches×5 inches. The right and left upper ends are formed in such a way that they jut out from the belt pocket into the belt area, in order to ensure a better fit.

Also sewn onto the front is an additional pocket, which holds the plastic breadth in the middle of the polyester cushion, and thus e.g. prevents the edge of the shell from pressing into the skin when seated.

The cherry stone cushion can be warmed up in the kitchen oven or in the microwave. In this way, it serves not only to absorb kicks, but it also helps to provide relaxation, as it pleasantly warms the abdomen of the mother as well as the feet of the baby.

In order to achieve an optimal shock absorbing and warming effect, it should be filled with at least approx. 170 g of dried cherry pits.

Furthermore, the warm cushion may be filled with other natural fillings, which are capable of storing heat. The following filling materials are named as examples: rye, spelt corn, rapeseed und grape seeds.

The dimensions of the cherry pit cushions should be reconciled with the belt pocket, and in particular with the additional pocket attached to the polyester cushion. The preferred dimensions are 8 inches×5 inches.

Depending on how far the Caesarean scar has already healed, some mothers feel that cooling of the scar is more pleasant than a warming effect. In this case, a hot-cold compress is particularly practical, as it is suitable for both warming and cooling the abdomen and providing protection to the scar. The thickness of the hot-cold compress should be about 1 to 2 inches, in other words somewhat thicker than a conventional compress, so that it can adequately absorb the kicks of the baby. The temperature of the compress can be adjusted by storing it in the ice compartment or warming it up in hot water.

The mother also has the choice of applying warmth to the scar, cooling it or just using plain protection.

The invention has now been described in detail for purposes of clarity and understanding.

However, it will be appreciated that certain changes and modifications may be practiced within the scope of the appended claims.

1. C-section belt, consisting of a belt, which is to be placed around the abdomen of the mother, and a shock absorbing element, which is then situated on the upper side of the abdomen.

2. C-section belt according to claim 1, with which the shock absorbing element is a plastic shell.

3. C-section belt according to claim 1, with which the shock absorbing element is a foam material cushion.

4. C-section belt according to claim 1, with which the shock absorbing element is a polyester cushion.

5. C-section belt according to claim 1, with which the shock absorbing element is a cushion filled with cherry stones.

6. C-Section Belt according to claim 1, in which the shock absorbing element is a corn-filled cushion.

7. C-section belt according to claim 1, with which the shock absorbing element is a hot-cold compress which is filled with a gel suitable for warming or cooling.

8. C-section belt according to one of the preceding claims, with which the belt has a pocket which holds the shock absorbing element.

9. C-section belt according to one of the preceding claims, with which the belt and the shock absorbing element have a hight of at least 4 inches.

10. C-section belt according to one of the preceding claims, with which flexible elastic bands are sewn into the belt.

11. C-section belt according to one of the preceding claims, with which the shock absorbing elements have in each case a thickness of less than 2 inches.
